(19) United States
(12) Patent Application Publication
(10) Pub. No.: US 2018/0133154 A1
(43) Pub. Date: May 17, 2018

(54) DECOY NUCLEIC ACID CATIONIC LIPOSOME CARRIER AND PREPARATION METHOD THEREOF

(71) Applicants: JIANGSU KEYGEN BIOTECH CORP.,LTD, Nanjign, Jiangsu (CN); NANJING CORE TECH CO., LTD, Nanjing, Jiangsu (CN)

(72) Inventors: Yang Xiao, Nanjing, Jiangsu (CN); Jinlong Cui, Nanjing, Jiangsu (CN); Zhengrong Li, Nanjing, Jiangsu (CN); Qing Ye, Nanjing, Jiangsu (CN); Lingyun He, Nanjing, Jiangsu (CN); Xuegen Wang, Nanjing, Jiangsu (CN)

(21) Appl. No.: 15/572,845

(22) PCT Filed: Jul. 8, 2015
(86) PCT No.: PCT/CN2015/083577

§ 371 Date: Nov. 9, 2017

(30) Foreign Application Priority Data
May 22, 2015 (CN) 201510268568.6

(51) Int. Cl. A61K 9/127; A61K 31/711

(52) U.S. Cl. CPC A61K 9/127; A61K 31/711

(57) ABSTRACT

A preparation method of a Decoy nucleic acid cationic liposome carrier including: (1) mixing dioleoyl phosphoethanolamine and (2,3-dioleoyl-propyl)-trimethylamine in a mass ratio of 4:1 to 1:4, and adding an organic solvent to obtain a mixed solution through dissolution; (2) completely evaporating the organic solvent in the mixed solution obtained in step (1), dissolving the remaining solid fraction by using an HEPES buffer solution, firstly hydrating the solution for 30 to 60 min, and then ultrasonically processing the solution for 30 to 60 min; (3) filtering the mixed system obtained after processing in the step (2) by a membrane of 0.4 to 0.8 μm, then filtering the mixed system by a membrane of 0.03 to 0.2 μm, and preparing uniformly to distributed blank liposome with small grain size; and (4) mixing the blank liposome with protamine and Decoy nucleic acid according to a mass ratio of (50 to 120):(10 to 20):1, and incubating for 12 to 24 h at the temperature of 2° C. to 8° C. to form the complete Decoy nucleic acid cationic liposome carrier.

### TECHNICAL FIELD

The present invention belongs to the technical field of pharmaceutical preparation, and more particularly, relates to a Decoy nucleic acid cationic liposome carrier and a preparation method thereof.

### BACKGROUND

A lipidosome is a superfine particle with a diameter of a few nanometers and even a few micrometers formed by a phospholipid bilayer in direction arrangement. The interior and the exterior of the bilayer are respectively encapsulated by fat soluble and water soluble drugs. The lipidosome has the features of making the drug have a targeting ability, improving and extending the curative effect, alleviating the toxicity, avoiding the tolerance and changing the drug delivery route. When Rahman et al, firstly applied the lipidosome as a drug carrier since 1960s, the studies to the preparation technology, action mechanism, body distribution, pharmacology and toxicology, and other properties of the lipidosome have been continuously deepened.

The lipidosome may be divided into a neutral lipidosome, an electropositive lipidosome and an electronegative lipidosome according to the properties of the charges carried. Since the lipidosome has a structure similar to a biological membrane which has high safety, and can be absorbed around a target cell for a long time to promote the permeation and absorption of the drug, and may enter the cell through a fusion activity to release the drug, and may be connected with a heterogenicligand easily to obtain an active targeting effect in the meanwhile, the lipidosome has great potential in targeting drug delivery treatment while being served as a drug carrier, and has the advantages of increasing the appetency with a cancer cell, overcoming the tolerance, increasing the intake of the cancer cell to the drug, reducing the drug dosage, increasing the curative effect and having less toxic and side effects. The existing studies on the lipidosome mainly remain in how to deliver a target object into the cell by the lipidosome and how to reduce the cell toxicity while increasing the stability and encapsulation efficiency of the lipidosome.

The difficulty of penetrating into the nucleus is a technical problem that perplexes the Decoy nucleic acid drugs over time. The Decoy nucleic acid drug can control a genetic expression from a transcriptional level using a transcription factor as a target spot, and is a new drug with strong targeting ability, but has a major problem that a drug delivery system needs to carry the drug to penetrate through a cell membrane and a cell nucleus since a target spot transcription factor affected by the Decoy drug usually exists in the cell nucleus. Reports about the lipidosome helping the drug to enter the cell nucleus have not been found yet in the prior art.

### SUMMARY

The technical problem to be solved by the present invention is to provide a Decoy nucleic acid cationic liposome carrier to increase a membrane penetrating rate and a nucleus penetrating rate when the cell is transfected.

The present invention also needs to provide a preparation method of the above-mentioned Decoy nucleic acid cationic liposome carrier.

In order to solve the technical problem above, the following technical solutions are used in the present invention.

1. A preparation method of a Decoy nucleic acid cationic liposome carrier comprises the following steps of:

(1) mixing dioleoyl phosphoethanolamine and (2,3-dioleoyl-propyl)-trimethylamine in a mass ratio of 4:1 to 1:4, and adding an organic solvent to obtain a mixed solution through dissolution;

(2) completely evaporating the organic solvent in the mixed solution obtained in step (1), dissolving the remaining solid fraction by using an HEPES buffer solution, firstly hydrating the solution for 30 to 60 min, and then ultrasonically processing the solution for 30 to 60 min;

(3) filtering the mixed system obtained after processing in the step (2) by a membrane of 0.4 to 0.8 μm, and then filtering the mixed system by a membrane of 0.03 to 0.2 μm, to prepare a uniformly distributed blank liposome with a small grain size;

(4) mixing the blank liposome with protamine and Decoy nucleic acid according to a mass ratio of (50 to 120):(10 to 20):1, and incubating for 12 to 24 h at the temperature of 2° C. to 8° C. to form the complete Decoy nucleic acid cationic liposome carrier.

In step (1), the organic solvent is trichloromethane.

In step (1), 10 ml organic solvent is added into per 1 mg mixture of dioleoyl phosphoethanolamine and (2,3-dioleoyl-propyl)-trimethylamine.

In step (2), the method of evaporating the organic solvent is to use a rotary evaporator to evaporate.

Preferably, in step (2), the HEPES buffer solution is 3 to 5 mol/L HEPES buffer solution with a pH of 7.4.

Preferably, in step (2), the hydration temperature is 20° C. to 30° C. and the ultrasound power is 100 to 200 W.

In step (3), the membrane is a polycarbonate membrane, and the mixed system is firstly filtered by a membrane of 0.4 to 0.8 μm for 10 to 20 times, and then is filtered by a membrane of 0.03 to 0.1 μm for 10 to 20 times.

A Decoy nucleic acid cationic liposome carrier obtained by the preparation method above also falls into the protection scope of the present invention.

An application of the Decoy nucleic acid cationic liposome carrier above for carrying a drug to penetrate through a cell membrane and a cell nucleus in a drug delivery system also falls into the protection scope of the present invention.

Beneficial effect: compared with the most common transfection reagent lipo2000 in the current market, the membrane penetrating rate of the present intention is increased by 30% and the nucleus penetrating rate thereof is increased by 90% when hek293 is used as a transfection cell; compared with the lipo3000 with the best transfection efficiency in the market, the transfection effect of the present invention is basically the same, and meanwhile, the present invention has no cell toxicity.

### BRIEF DESCRIPTION OF THE DRAWINGS

FIG. 1: A is a DOPE structure and B is a structural schematic diagram of DOTAP;

FIG. 2: a schematic diagram of a nucleic acid:LMWP:lipidosome compound, wherein A is a lipidosome layer, B is DNA and C is LMWP; dDNA is a distance between axles of two DNA molecules, δw is the thickness of the lipidosome layer, and δm is the thickness of the DNA molecule;

FIG. 3A is an average grain size detection diagram of a cationic liposome in embodiment 1;

FIG. 3B is a detection result diagram of zeta potential of the cationic liposome in embodiment 1;

FIG. 4A is a transfection effect diagram of lipo2000;

FIG. 4B is a transfection effect diagram of lipo3000;

FIG. 4C is a transfection effect diagram of a Decoy nucleic acid cationic liposome containing 250 ng DNA;

FIG. 4D is a transfection effect diagram of a Decoy nucleic acid cationic liposome containing 500 ng DNA;

FIG. 4E is a transfection effect diagram of a Decoy nucleic acid cationic liposome containing 1000 ng DNA;

FIG. 5A is a nucleus penetrating effect diagram of lipo2000;

FIG. 5B is a nucleus penetrating effect diagram of lipo3000;

FIG. 5C is a nucleus penetrating effect diagram of the Decoy nucleic acid cationic liposome containing 250 ng DNA;

FIG. 5D is a nucleus penetrating effect diagram of the Decoy nucleic acid cationic liposome containing 500 ng DNA; and

FIG. 5E is a nucleus penetrating effect diagram of the Decoy nucleic acid cationic liposome containing 1000 ng DNA.

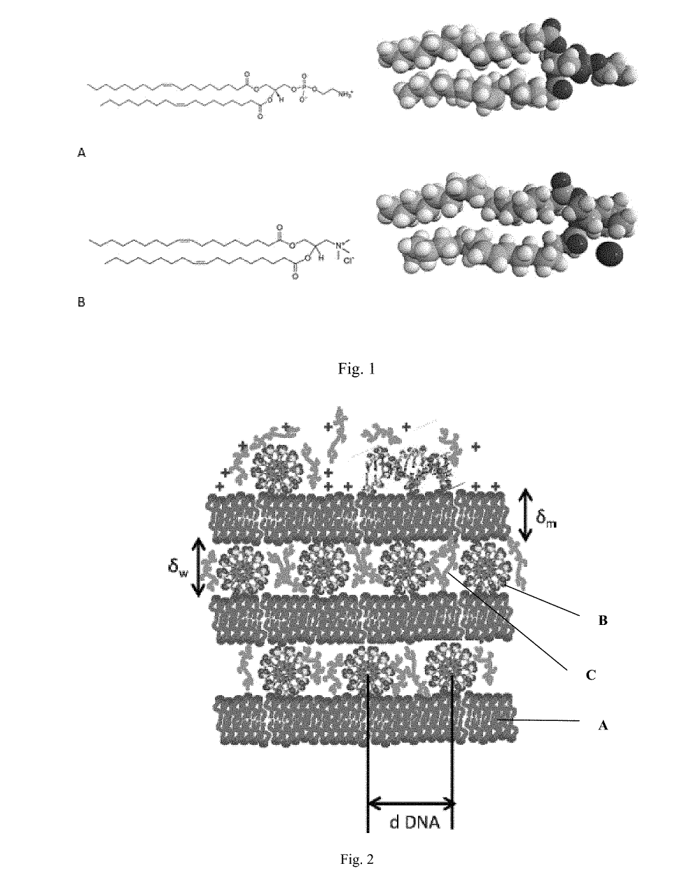

### DETAILED DESCRIPTION

The present invention will be better understood according to the following embodiments. However, it is easy for those skilled in the art to understand that the contents described in the embodiments are only used for describing the present invention, which shall not and will not limit the present invention described in details in the claims.

### Embodiment 1 Preparation of a Decoy Nucleic Acid Cationic Liposome

(1) 3 mg DOPE and 3 mg DOTAP were dissolved in 20 ml trichloromethane to prepare a lipid solution.

(2) The lipid solution above was added into a round-bottom flask for rotary reduced pressure vaporization in a 20° C. thermostatic waterbath, adjustment to the rotation speed and the temperature was concerned to avoid the generation of bubbles, an organic solvent was removed to form a lipid membrane, 3 ml 4 mM HEPES with a pH of 7.4 was added into the round-bottom flask above to subject to hydration under 20° C. for 30 min and then ultrasounding for 30 min after hydration so as to prepare a rough lipidosome solution, wherein the ultrasonic power was 100 W; the rough lipidosome solution was filtered by a membrane of 0.4 μm for 10 times, and then filtered by a membrane of 0.2 μm for 10 times to prepare 2 mg/ml lipidosome solution.

(4) 2 mg protamine and 1 mg Decoy nucleic acid were respectively dissolved in 3 ml 4 mM HEPES buffer solution with a pH of 7.4 to prepare a protamine and Decoy nucleic acid solution.

(4) 670 μl 4 mM HEPES buffer solution with a pH of 7.4, 275 μl lipidosome solution, 50 LMWP solution and 5 μl Decoy nucleic acid solution were added into a 1.5 ml eppendorf test tube in sequence to form a mixed solution of Decoy nucleic acid cationic liposome with a mass ratio of 110:20:1, the mixed solution was placed into a 4° C. refrigerator for static incubation for 12 h to obtain the Decoy nucleic acid cationic liposome.

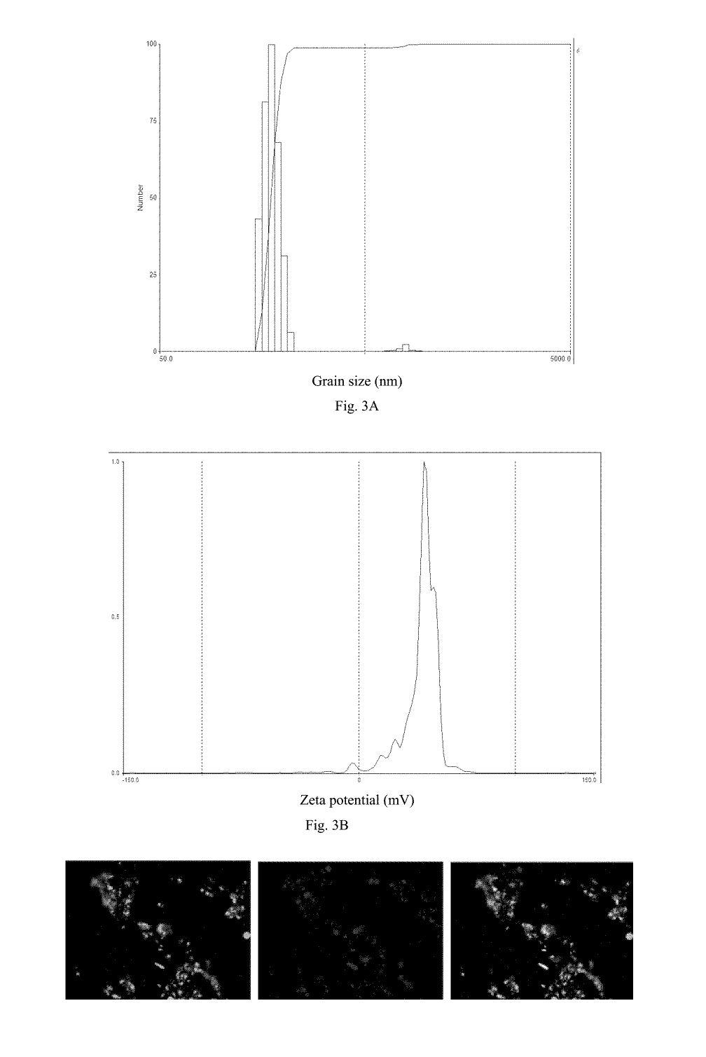

The prepared Decoy nucleic acid cationic liposome was detected by a granulometer and a Zeta potential instrument, and the detection results were shown in FIGS. 3A and 3B, wherein the average grain size was 200 nm and the zeta potential was 38.45 mV.

### Embodiment 2 Preparation of a Decoy Nucleic Acid Cationic Liposome

(1) 3 mg DOPE and 3 mg DOTAP were dissolved in 20 ml trichloromethane to prepare a lipid solution.

(2) The lipid solution above was added into a round-bottom flask for rotary reduced pressure vaporization in a 20° C. thermostatic waterbath, adjustment to the rotation speed and the temperature was concerned to avoid the generation of bubbles, an organic solvent was removed to form a lipid membrane, 3 ml 4 mM HEPES with a pH of 7.4 was added into the round-bottom flask above to subject to hydration under 20° C. for 30 min and then ultrasounding for 30 min after hydration so as to prepare a rough lipidosome solution, wherein the ultrasonic power was 100 W; the rough lipidosome solution was filtered by a membrane of 0.4 μm for 10 times and then filtered by a membrane of 0.2 μm for 10 times to prepare 2 mg/ml lipidosome solution.

(4) 2 mg protamine and 1 mg Decoy nucleic acid were respectively dissolved in 1 ml 4 mM HEPES buffer solution with a pH of 7.4 to prepare a protamine and Decoy nucleic acid solution.

(4) 845 μl 4 mM HEPES buffer solution with a pH of 7.4, 125 μl lipidosome solution, 25 LMWP solution and 5 μl Decoy nucleic acid solution were added into a 1.5 ml eppendorf test tube in sequence to form a mixed solution of Decoy nucleic acid cationic liposome with a mass ratio of 50:10:1, the mixed solution was placed into a 4° C. refrigerator for static incubation for 12 h to obtain the Decoy nucleic acid cationic liposome.

The prepared Decoy nucleic acid cationic liposome was detected by a granulometer and a Zeta potential instrument, the average grain size was 157.7 nm and the zeta potential was 29.15 mV.

### Embodiment 3 Preparation of a Decoy Nucleic Acid Cationic Liposome

(1) 3 mg DOPE and 12 mg DOTAP were dissolved in 20 ml trichloromethane to prepare a lipid solution.

(2) The lipid solution above was added into a round-bottom flask for rotary reduced pressure vaporization in a 20° C. thermostatic waterbath, adjustment to the rotation speed and the temperature was concerned to avoid the generation of bubbles, an organic solvent was removed to form a lipid membrane, 3 ml 4 mM HEPES with a pH of 7.4 was added into the round-bottom flask above to subject to hydration under 20° C. for 30 min and then ultrasounding for 30 min after hydration so as to prepare a rough lipidosome solution, wherein the ultrasonic power was 100 W; the rough lipidosome solution was filtered by a membrane of 0.4 μm for 10 times, and then filtered by a membrane of 0.2 μm for 10 times to prepare 2 mg/ml lipidosome solution.

(4) 2 mg protamine and 1 mg Decoy nucleic acid were respectively dissolved in 1 ml 4 mM HEPES buffer solution with a pH of 7.4 to prepare a protamine and Decoy nucleic acid solution.

(4) 670 μl 4 mM HEPES buffer solution with a pH of 7.4, 300 μl lipidosome solution, 25 μl LMWP solution and 5 μl Decoy nucleic acid solution were added into a 1.5 ml eppendorf test tube in sequence to form a mixed solution of Decoy nucleic acid cationic liposome with a mass ratio of 120:10:1, the mixed solution was placed into a 4° C. refrigerator for static incubation for 12 h to obtain the Decoy nucleic acid cationic liposome.

The prepared Decoy nucleic acid cationic liposome was detected by a granulometer and a Zeta potential instrument, the average grain size was 188.9 nm and the zeta potential was 27.14 mV.

### Embodiment 4 Preparation of a Decoy Nucleic Acid Cationic Liposome was as Follows

(1) 12 mg DOPE and 3 mg DOTAP were dissolved in 20 ml trichloromethane to prepare a lipid solution.

(2) The lipid solution above was added into a round-bottom flask for rotary reduced pressure vaporization in a 20° C. thermostatic waterbath, adjustment to the rotation speed and the temperature was concerned to avoid the generation of bubbles, an organic solvent was removed to form a lipid membrane, 3 ml 4 mM HEPES with a pH of 7.4 was added into the round-bottom flask above to subject to hydration under 20° C. for 30 min and then ultrasounding for 30 min after hydration so as to prepare a rough lipidosome solution, wherein the ultrasonic power was 100 W; the rough lipidosome solution was filtered by a membrane of 0.4 μm for 10 times, and then filtered by a membrane of 0.2 μm for 10 times to prepare 2 mg/ml lipidosome solution.

(4) 2 mg protamine and 1 mg Decoy nucleic acid were respectively dissolved in 1 ml 4 mM HEPES buffer solution with a pH of 7.4 to prepare a protamine and Decoy nucleic acid solution.

(4) 820 μl 4 mM HEPES buffer solution with a pH of 7.4, 125 μl lipidosome solution, 50 μl LMWP solution and 5 μl Decoy nucleic acid solution were added into a 1.5 ml eppendorf test tube to form a mixed solution of Decoy nucleic acid cationic liposome with a mass ratio of 50:20:1, the mixed solution was placed into a 4° C. refrigerator for static incubation for 12 h to obtain the Decoy nucleic acid cationic liposome.

The prepared Decoy nucleic acid cationic liposome was detected by a granulometer and a Zeta potential instrument, the average grain size was 210.1 nm and the zeta potential was 20.14 mV.

### Embodiment 5: Comparison Between the Transfection and Nucleus Penetrating Ability of Decoy Nucleic Acid Cationic Liposome and the Transfection and Nucleus Penetrating Ability of Lipo2000 and Lipo3000

DOPE to DOTAP lipidosome, LMWP (protamine) and DNA STAT3 Decoy ODN were prepared according to a mass ratio of 110:20:1 by the method provided in embodiment 1, wherein the 5′ end of the DNA was labeled with cy3 in advance. A HEK293 cell was inoculated to a 6-hole culture plate fixed by polyarginine in advance, and a transfection operation was conducted when the production of the cell reached to 50% saturability. The transfection reagent was diluted by serum-free and dual resistance-free Opti-MEM culture mediums of various amounts.

Wherein:

the experimental group of lipo2000: 198 μl OMEM culture medium, 1.5 μl lipo2000 reagent and 0.5 μl 1 mg/ml DNA;

the experimental group of lipo3000: 197 μl OMEM culture medium, 1 μl P3000 reagent, 1.5 μl lipo3000 reagent and 0.5 μl 1 mg/ml DNA; and

the experimental group of Decoy nucleic acid cationic liposome: 250 ng, 500 ng and 1000 ng Decoy nucleic acid cationic liposome prepared according to embodiment 1 were respectively mixed with the OMEM culture mediums of various amounts.

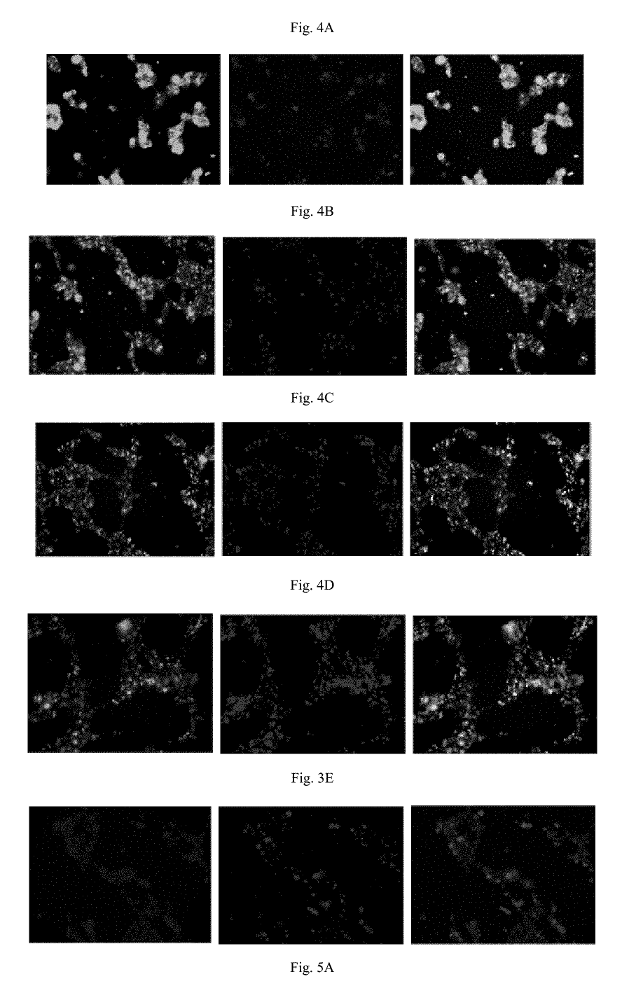

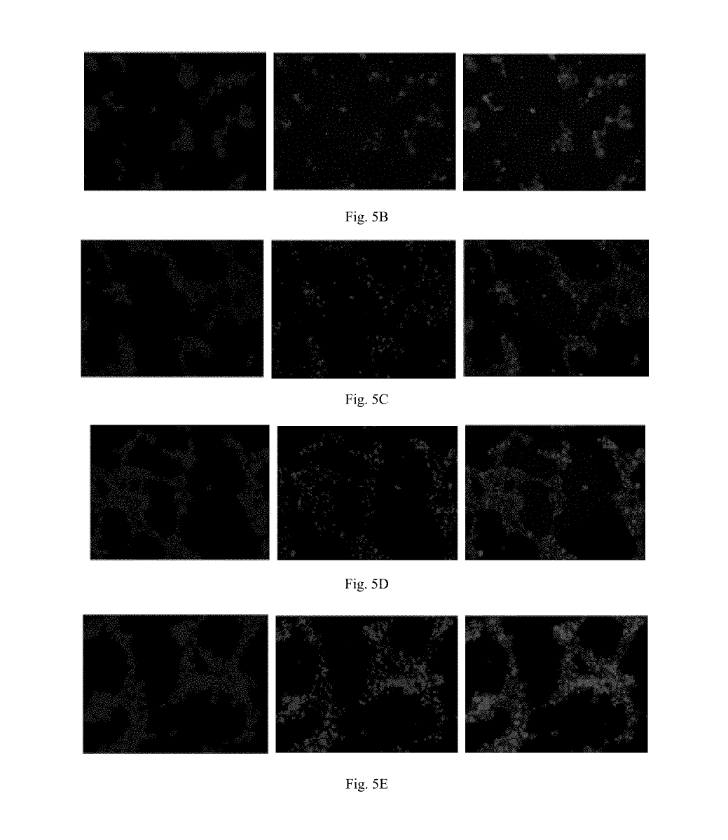

Each experimental group was added into a6-hole plate cell for incubation under 37° C. for 16 h, the cell supernatant was taken away, the cell membrane was dyed by Dio reagent, and the cell nucleus was dyed by Hochest reagent, which was photographed by an ImageXpress® Micro high content imaging system, and the transfection rate and the nucleus penetrating rate were calculated. The results were as shown in FIGS. 4A to 4E, and FIGS. 5A to 5E. Wherein, FIGS. 4A to 4E were the transfection effect diagrams of lipo2000, lipo3000, a Decoy nucleic acid cationic liposome containing 250 ng DNA, a Decoy nucleic acid cationic liposome containing 500 ng DNA, and a Decoy nucleic acid cationic liposome containing 1000 ng DNA respectively, the figures were, from left to right, the effect diagram of the cell membrane dyed by Dio reagent, the distribution diagram of the Decoy nucleic acid labeled by 5′cy3, and the cell transfection effect diagram obtained by overlapping the figures above respectively; and FIGS. 5A to 5E were the nucleus penetrating effect diagrams of the lipo2000, the lipo3000, the Decoy nucleic acid cationic liposome containing 250 ng DNA, the Decoy nucleic acid cationic liposome containing 500 ng DNA, and the Decoy nucleic acid cationic liposome containing 1000 ng DNA respectively, and the figures were, from left to right, the effect diagram of the cell nucleus dyed by Hochest reagent, the distribution diagram of the Decoy nucleic acid labeled by 5′cy3, and the nucleus penetrating effect diagram obtained by overlapping the two figures above respectively.

The results showed that the transfection rate of Lipo2000 was 65.6% and the nucleus penetrating rate thereof was 28.8%, the transfection rate of Lipo3000 was 89.8% and the nucleus penetrating rate thereof was 61.8%, the transfection rate of the Decoy nucleic acid cationic liposome containing 250 ng DNA was 66.3% and the nucleus penetrating rate thereof was 29.5%, the transfection rate of the Decoy nucleic acid cationic liposome containing 500 ng DNA was 85.5%% and the nucleus penetrating rate thereof was 54.0%, the transfection rate of the Decoy nucleic acid cationic liposome containing 1000 ng DNA was 98.9%, and the nucleus penetrating rate thereof was 76.9%.

1. A preparation method of a Decoy nucleic acid cationic liposome carrier, comprising the following steps of:
(1) mixing dioleoyl phosphoethanolamine and (2,3-dioleoyl-propyl)-trimethylamine in a mass ratio of 4:1 to 1:4, and adding an organic solvent to obtain a mixed solution through dissolution;
(2) completely evaporating the organic solvent in the mixed solution obtained in step (1), dissolving the remaining solid fraction by using an HEPES buffer solution, firstly hydrating the solution for 30 to 60 min, and then ultrasonically processing the solution for 30 to 60 min,
(3) filtering the mixed system obtained after processing in the step (2) by a membrane of 0.4 to 0.8 μm, and then filtering the mixed system by a membrane of 0.03 to 0.2 μm, to prepare a uniformly distributed blank liposome with a small grain size;
(4) mixing the blank liposome with protamine and Decoy nucleic acid according to a mass ratio of (50 to 120):(10 to 20):1, and incubating for 12 to 24 h at the temperature of 2° C. to 8° C. to form the complete Decoy nucleic acid cationic liposome carrier.

2. The preparation method of a Decoy nucleic acid cationic liposome carrier according to claim 1, wherein in step (1), the organic solvent is trichloromethane.

3. The preparation method of a Decoy nucleic acid cationic liposome carrier according to claim 1, wherein in step (1), 10 ml organic solvent is added into per 1 mg mixture of dioleoyl phosphoethanolamine and (2,3-dioleoyl-propyl)-trimethylamine.

4. The preparation method of a Decoy nucleic acid cationic liposome carrier according to claim 1, wherein in step (2), the method of evaporating the organic solvent is to use a rotary evaporator to evaporate.

5. The preparation method of a Decoy nucleic acid cationic liposome carrier according to claim 1, wherein in step (2), the HEPES buffer solution is 3 to 5 mol/L HEPES buffer solution with a pH of 7.4.

6. The preparation method of a Decoy nucleic acid cationic liposome carrier according to claim 1, wherein in step (2), the hydration temperature is 20° C. to 30° C. and the ultrasound power is 100 to 200 W.

7. The preparation method of a Decoy nucleic acid cationic liposome carrier according to claim 1, wherein in step (3), the membrane is a polycarbonate membrane, and the mixed system is firstly filtered by a membrane of 0.4 to 0.8 μm for 10 to 20 times, and then is filtered by a membrane of 0.03 to 0.1 μm for 10 to 20 times.

8. A Decoy nucleic acid cationic liposome carrier obtained by the preparation method according to claim 1.

9. An application of the Decoy nucleic acid cationic liposome carrier according to claim 8 for carrying a drug to penetrate through a cell membrane and a cell nucleus in a drug delivery system.

10. The application according to claim 9, wherein the drug is a Decoy nucleic acid drug.
